(19) United States
(12) Patent Application Publication
(10) Pub. No.: US 2008/0166804 A1
(43) Pub. Date: Jul. 10, 2008

(54) Compostions and methods for growing human embryonic stem cells

(76) Inventors: Michael J. Shamblott, Baltimore, MD (US); Michael Cohen, West Orange, NJ (US)

Correspondence Address: Pearl Cohen Zedek Latzer, LLP, 1500 Broadway, 12th Floor, New York, NY, 10036, US

(21) Appl. No.: 11/826,539

(22) Filed: Jul. 16, 2007

Related U.S. Application Data
(60) Provisional application No. 60/830,668, filed on Jul. 14, 2006.

(51) Int. Cl. C12N 5/06

(52) U.S. Cl. 435/366; 435/377; 435/378

(57) ABSTRACT

Methods for deriving and cultivating human embryonic stem (ES) cells and maintaining their pluripotency in culture is provided by utilizing secreted products obtained from the culture medium of human embryonic germ (EG) cell derivatives, such as embryoid body-derived cells. Substrates include compounds such as collagen I, fibronectin, or superfibronectin, or extracellular matrix, typically human derived.

### CROSS-REFERENCE TO RELATED APPLICATION

This application claims priority to U.S. provisional patent application Ser. No. 60/830,668, filed Jul. 14, 2006, which is incorporated herein by reference in its entirety.

### BACKGROUND OF THE INVENTION

Embryonic germ (EG) cells are pluripotent stem cells derived from primordial germ cells that arise in the late embryonic and early fetal period. EG cells have been derived from several species, including mouse [Matsui, Y., D. Toksoz, S. Nishikawa, S. Nishikawa, D. Williams, K. Zsebo, and B. L. Hogan, (1991) Effect of Steel factor and leukaemia inhibitory factor on murine primordial germ cells in culture. Nature. 353: p. 750-2; Resnick, J. L., L. S. Bixler, L. Cheng, and P. J. Donovan, (1992) Long-term proliferation of mouse primordial germ cells in culture. Nature. 359: p. 550-1], pig [Piedrahita, J. A., K. Moore, B. Oetama, C. K. Lee, N. Scales, J. Ramsoondar, F. W. Bazer, and T. Ott, (1998) Generation of transgenic porcine chimeras using primordial germ cell-derived colonies. Biol Reprod. 58: p. 1321-9], chicken [Park, T. S. and J. Y. Han, (2000) Derivation and characterization of pluripotent embryonic germ cells in chicken. Mol Reprod Dev. 56: p. 475-82], and human [Shamblott, M. J., J. Axelman, S. Wang, E. M. Bugg, J. W. Littlefield, P. J. Donovan, P. D. Blumenthal, G. R. Huggins, and J. D. Gearhart, (1998) Derivation of pluripotent stem cells from cultured human primordial germ cells. Proc Natl Acad Sci USA. 95: p. 13726-31; Park, J. H., S. J. Kim, J. B. Lee, J. M. Song, C. G. Kim, S. Roh, 2nd, and H. S. Yoon, (2004) Establishment of a human embryonic germ cell line and comparison with mouse and human embryonic stem cells. Mol Cells. 17: p. 309-15; Li, X. H., H. C. Cong, Z. Wang, C. F. Wu, and Y. L. Cao, (2002) Isolation and culture of human pluripotent embryonic germ cells. Shi Yan Sheng Wu Xue Bao. 35: p. 142-6; Turnpenny, L., S. Brickwood, C. M. Spalluto, K. Piper, I. T. Cameron, D. I. Wilson, and N. A. Hanley, (2003) Derivation of human embryonic germ cells: an alternative source of pluripotent stem cells. Stem Cells. 21: p. 598-609]. Like embryonic stem (ES) cells, EG cells differentiate in vitro to form complex cell aggregates termed embryoid bodies (EBs), which are comprised of mature cell types from many different cell lineages and rapidly proliferating precursor/progenitor cells.

Enzymatic disaggregation of human EG-derived EBs and outgrowth of resulting cells yields embryoid body-derived (EBD) cell cultures. EBD cultures and clonal cell lines proliferate robustly with a normal diploid karyotype and express a broad range of precursor, progenitor and terminally differentiated markers from developmentally distinct cell lineages [Shamblott, M., J. Axelman, J. Littlefield, P. Blumenthal, G. Huggins, Y. Cui, L. Cheng, and J. Gearhart, (2001) Human embryonic germ cell derivatives express a broad range of developmentally distinct markers and proliferate extensively in vitro. Proc Natl Acad Sci USA. 98: p. 113-118]. EBD cultures are named such that the first two letters refer to the EG culture from which it was derived, the third letter indicates the growth media in which it was derived and is maintained and the fourth letter indicates the matrix on which it is grown.

Following transplantation, cells from EBD culture SDEC have partially restored motor function to rats paralyzed following infection by Sindbis virus [Kerr, D. A., J. Llado, M. J. Shamblott, N. J. Maragakis, D. N. Irani, T. O. Crawford, C. Krishnan, S. Dike, J. D. Gearhart, and J. D. Rothstein, (2003) Human embryonic germ cell derivatives facilitate motor recovery of rats with diffuse motor neuron injury. J Neurosci. 23: p. 5131-40], partially restored the complement of striatal neurons in mice following excitotoxic lesion [Mueller, D., M. J. Shamblott, H. E. Fox, J. D. Gearhart, and L. J. Martin, (2005) Transplanted human embryonic germ cell-derived neural stem cells replace neurons and oligodendrocytes in the forebrain of neonatal mice with excitotoxic brain damage. J Neurosci Res. 82: p. 592-608] and participate in the regeneration of rat bladder following injury [Frimberger, D., N. Morales, M. Shamblott, J. D. Gearhart, J. P. Gearhart, and Y. Lakshmanan, (2005) Human embryoid body-derived stem cells in bladder regeneration using rodent model. Urology. 65: p. 827-32; Kim, M. S., N. S. Hwang, J. Lee, T. K. Kim, K. Leong, M. J. Shamblott, J. Gearhart, and J. Elisseeff, (2005) Musculoskeletal differentiation of cells derived from human embryonic germ cells. Stem Cells. 23: p. 113-23].

Embryonic stem (ES) cells are derived from the inner cell mass of preimplantation embryos [Evans, M. J. and M. H. Kaufman (1981). Establishment in culture of pluripotential cells from mouse embryos. Nature 292(5819): 154-6; Martin, G. R. (1981). Isolation of a pluripotent cell line from early mouse embryos cultured in media conditioned by teratocarcinoma stem cells. Proc. Natl. Acad. Sci. USA 78: 7634-7638; Thomson, J. A., J. Itskovitz-Eldor, et al. (1998). Embryonic stem cell lines derived from human blastocysts. Science 282(5391): 1145-7]. ES cells are pluripotent and are capable of differentiating into cells derived from all three embryonic germ layers. The traditional method used to derive mouse and human embryonic stem (ES) cells involves the use of support cells termed feeder cells or layers. These support cells provide a poorly understood set of signals that promote the conversion from blastocyst inner cell mass (ICM) cells to proliferating ES cells. Most commonly, primary cultures of mouse embryo fibroblasts are used as support cells for both mouse and human ES cultures. The requirement for support cells is not lost following derivation, and ES cell cultures are most commonly maintained on feeder layers until differentiation is desired. Since the signals supplied by support cells are not understood, it has been difficult to find substitute cell types or to remove cells altogether. For research purposes, support cells provide a source of experimental variability and cellular contamination to ES cultures but are not disabling in their impact.

However, a major obstacle to the use of ES cells for human therapy is the requirement for feeder cells, whether human or non-human. Human feeder layers potentially contaminate ES cells with allogeneic proteins or living cells, and the potential for contamination by infectious agents exists. Similar undesirable properties exist when non-human feeder cells are used. Eliminating feeder cells has not been successful. When cultured in a standard culture environment in the absence of mouse embryonic fibroblasts as feeder cells, ES cells rapidly differentiate or fail to survive. Attempts have been made to replace the feeder or support cells using cell-free components or at least avoid non-human components or cells. While some replacements have shown short-term promising results, such attempts have proven insufficient to support robust, continued propagation. For example, WO/9920741 describes the growth of ES cells in a nutrient serum effective to support the growth of primate-derived primordial stem cells and a substrate of feeder cells or an extracellular matrix component derived from feeder cells. The medium further includes non-essential amino acids, an anti-oxidant, and growth factors that are either nucleosides or a pyruvate salt. U.S. Pat. No. 6,642,048 reports growth of ES cells in feeder-free culture, using conditioned medium from such cells. U.S. Pat. No. 6,800,480 describes a cell culture medium for growing primate-derived primordial stem cells comprising a low osmotic pressure, low endotoxin basic medium comprising a nutrient serum and an extracellular matrix derived from the feeder cells. The medium further includes non-essential amino acids, an anti-oxidant (for example, beta-mercaptoethanol), and, optionally, nucleosides and a pyruvate salt. Need exists for better medium that supports the long-term propagation of ES cells in a pluripotent state.

### SUMMARY OF THE INVENTION

In one embodiment, a method for cultivating human embryonic stem (ES) cells and maintaining the pluripotency thereof is provided comprising growing the human embryonic stem (ES) cells in a culture medium comprising secreted products from human embryonic germ (EG) cell derivatives. In another embodiment, the human embryonic germ (EG) cell derivatives are embryoid body-derived cells (EBD), such as but not limited to cell culture LVEC or SDEC. In another embodiment, a substrate is provided, such as collagen I, collagen IV, fibronectin, superfibronectin, laminin, heparan sulfate proteoglycan, entactin, or any combination thereof. Typically, the collagen I is bovine or human type 1 collagen. In another embodiment, the substrate comprises any synthetic or biosynthetic cell adhesion molecule or mixture thereof. In another embodiment, the substrate is extracellular matrix, such as that obtained from human embryonic germ (EG) cell derivatives, or from EHS mouse sarcoma basement membrane or from human extracellular matrix. In another embodiment, the substrate comprises any synthetic or biosynthetic cell adhesion molecule or a mixture thereof. Typically, the substrate is human derived.

In another embodiment, a composition for cultivating human stem cells and maintaining the pluripotency thereof is provided comprising secreted products from human embryonic germ (EG) cell derivatives, in combination with a substrate. In one embodiment, the human embryonic germ (EG) cell derivatives are human embryoid body-derived cells, such as but not limited to LVEC cells or SDEC cells. In another embodiment, the substrate is collagen 1, collagen IV, fibronectin, superfibronectin, laminin, heparan sulfate proteoglycan, entactin, or any combination thereof. Typically the substrate is human type I collagen. In another embodiment, the substrate comprises any synthetic or biosynthetic cell adhesion molecule or a mixture thereof. In another embodiment, the substrate is an extracellular matrix, such as but not limited to extracellular matrix is obtained from human embryonic germ (EG) cell derivatives, EHS mouse sarcoma basement membrane or human extracellular matrix. Typically, the substrate is human derived. In another embodiment, the human stem cells are human embryonic stem cells.

In another embodiment, a method is provided for obtaining a pluripotent human embryonic cell line comprising the steps of 1) isolating cells from the inner cell mass of a pre-implantation embryo; 2) introducing the cells of (1) into a culture medium comprising a composition as described above; and 3 ) growing the human embryonic stem cells that convert from inner cell mass cells over several passages in the culture medium, thereby obtaining a human embryonic stem cell line derived from the pre-implantation embryo. In one embodiment, the human embryonic germ (EG) cell derivatives are human embryoid body-derived cells, such as but not limited to LVEC cells or SDEC cells. In another embodiment, the substrate is collagen I, collagen IV, fibronectin, superfibronectin, laminin, heparan sulfate proteoglycan, entactin, or any combination thereof. Typically the substrate is human type I collagen. In another embodiment, the substrate comprises any synthetic or biosynthetic cell adhesion molecule or a mixture thereof. In another embodiment, the substrate is an extracellular matrix, such as but not limited to extracellular matrix is obtained from human embryonic germ (EG) cell derivatives, EHS mouse sarcoma basement membrane or human extracellular matrix. Typically the substrate is human derived.

In another embodiment, a kit is provided for cultivating human embryonic stem (ES) cells and maintaining the pluripotency thereof, the kit comprising a first container containing secreted products from human embryonic germ (EG) cell derivatives, a second container containing substrate, and instructions for the use thereof. In one embodiment, the human embryonic germ (EG) cell derivatives are human embryoid body-derived cells, such as but not limited to LVEC cells or SDEC cells. In another embodiment, the substrate is collagen I, collagen IV, fibronectin, superfibronectin, laminin, heparan sulfate proteoglycan, entactin, or any combination thereof. Typically the substrate is human type I collagen. In another embodiment, the substrate comprises any synthetic or biosynthetic cell adhesion molecule or a mixture thereof. In another embodiment, the substrate is an extracellular matrix, such as but not limited to extracellular matrix is obtained from human embryonic germ (EG) cell derivatives, EHS mouse sarcoma basement membrane or human extracellular matrix. Typically, the substrate is human derived.

In another embodiment, the invention is directed to a composition comprising pluripotent human embryonic stem (ES) cells and secreted products from human embryonic germ (EG) cell derivatives. In another embodiment, the human embryonic germ (EG) cell derivatives are human embryoid body-derived cells, such as but not limited to LVEC cells or SDEC cells. In another embodiment, the substrate is collagen I, collagen IV, fibronectin, superfibronectin, laminin, heparan sulfate proteoglycan, entactin, or any combination thereof. Typically the substrate is human type I collagen. In another embodiment, the substrate comprises any synthetic or biosynthetic cell adhesion molecule or a mixture thereof. In another embodiment, the substrate is an extracellular matrix, such as but not limited to extracellular matrix is obtained from human embryonic germ (EG) cell derivatives, EHS mouse sarcoma basement membrane or human extracellular matrix.

In another embodiment, cultured pluripotent human embryonic stem (ES) cells are provided that are obtained by the process of 1) providing a culture medium comprising secreted products from human embryonic germ (EG) cell derivatives, together with a substrate, 2) introducing human embryonic stem cells thereto; and 3) growing the human embryonic stem cells therein to produce cultured pluripotent human embryonic stem cells. In one embodiment, the human embryonic germ (EG) cell derivatives are human embryoid body-derived cells, such as but not limited to LVEC cells or SDEC cells. In another embodiment, the substrate is collagen I, collagen IV, fibronectin, superfibronectin, laminin, heparan sulfate proteoglycan, entactin, or any combination thereof. Typically the substrate is human type I collagen. In another embodiment, the substrate comprises any synthetic or biosynthetic cell adhesion molecule or a mixture thereof. In another embodiment, the substrate is an extracellular matrix, such as but not limited to extracellular matrix is obtained from human embryonic germ (EG) cell derivatives, EHS mouse sarcoma basement membrane or human extracellular matrix. Typically, the substrate is human derived.

In another embodiment, methods are provided to administer cell-based therapy using embryonic stem (ES) cells to a subject in need thereof by growing embryonic stem (ES) cells in accordance to the teachings herein then administering the embryonic stem (ES) cells to the subject.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

### BRIEF DESCRIPTION OF THE DRAWING

FIG. 1 shows the percent of embryonic stem (ES) cells that are OCT4 positive, an indication of pluripotency, when ES cells are grown under the following conditions: MCC, secreted products from mouse embryo fibroblasts and a substrate of type I collagen; SCC, secreted products from human embryonic germ (EG) cell derivatives on a substrate of type I collagen; MMC, secreted products from mouse embryo fibroblasts on a substrate of Matrigel; and SMC, secreted products from human embryonic germ (EG) cell derivatives on a Matrigel substrate. The corresponding population doublings in the four groups during the third passage are 0, 2.6, 2.9 and 2.4, respectively.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

While the therapeutic and other applications of embryonic stem (ES) cells are projected to have a major impact on the future of health care and the treatment of a large number of diseases, methods for deriving ES cells from the embryo and maintaining the pluripotency of thus-derived ES cells in a medium readily compatible with human administration has hindered progress in this field. While ES cell can be derived from blastocyst inner mass cells and maintained in a pluripotent state using mouse feeder cell layers and conditioned medium from mouse feeder cells, use of any mouse products at any point during the preparation of ES cells for human therapy has adverse regulatory implications. As embodied herein, in an effort to identify substitutes for mouse embryo fibroblasts in deriving and maintaining ES cells, is was found that secreted products from embryonic germ (EG) cell derivative cultures provided the necessary components to permit both the derivation and propagation of ES cells in the absence of any mouse-derived materials (including both cells and secreted products including extracellular matrix).

Thus, in one embodiment of the invention, a method is provided for cultivating human embryonic stem (ES) cells and maintaining the pluripotency thereof comprising growing the human embryonic stem (ES) cells in a culture medium comprising secreted products from human embryonic germ (EG) cell derivatives. The human embryonic germ (EG) cell derivatives typically are embryoid body-derived cells, as described in further detail below. Exemplary but non-limiting human embryoid body-derived cells (EBD) are LVEC cells or SDEC cells.

In one embodiment, the aforementioned method further comprises a substrate. The substrate can be, by way of non-limiting example, collagen I, collagen IV, fibronectin, superfibronectin, laminin, heparan sulfate proteoglycan, entactin, or any combination thereof. Typically, the collagen I is human type I collagen. Typically, a substrate of human origin is used in order to avoid the presence of non-human components in ES cultures, but for purposes other than human therapeutic uses, non-human components may be present. In another embodiment, the substrate comprises any synthetic or biosynthetic cell adhesion molecule or a mixture thereof.

The aforementioned substrates such as collagen I and fibronectin or superfibronectin can be purchased as purified proteins or proteoglycans from any number of suppliers (such as Sigma Chemical Company, Innovative Research or Research Diagnostics Inc.) or prepared and purified in the laboratory. Fibronectin is an extracellular matrix protein that is important in development, wound healing and tumorigenesis. In the blood it is dimeric, but in tissues forms disulphide crosslinked fibrils. Superfibronectin is derived using a fragment from the first type-III repeat of fibronectin which binds to fibronectin and induces spontaneous disulphide crosslinking of the molecule into multimers of high relative molecular mass which resemble matrix fibrils. Treatment of fibronectin with this inducing fragment converts fibronectin into a form that has greatly enhanced adhesive properties (hence the term superfibronectin) and which suppresses cell migration [Morla, A., et al. (1994). Superfibronectin is a functionally distinct form of fibronectin. Nature 367(6459): 193-6].

In addition to the aforementioned substrates, other synthetic or biosynthetic adhesion molecules can be used, including fragments and peptides from the aforementioned proteins that support growth of ES cells. Typically will be substrates that are human derived.

In another embodiment, the aforementioned method further comprises the use of an extracellular matrix. Extracellular matrix may be obtained from normal cells or immortalized cell lines. Non-limiting examples include extracellular matrix from human embryonic germ (EG) cell derivatives, such as from human embryoid body-derived cells. Non-limiting examples of such cells include LVEC cells or SDEC cells. In another embodiment, the extracellular matrix is EHS mouse sarcoma basement membrane or human extracellular matrix. As noted above, typically a human extracellular matrix is used in order to avoid the presence of non-human components in ES cultures, but for purposes other than human therapeutic uses, non-human components may be present. In addition to the examples above human extracellular matrix can be obtained from any human cell type.

In another embodiment, a composition is provided for cultivating human embryonic stem cells and maintaining the pluripotency thereof, the composition comprising secreted products from human embryonic germ (EG) cell derivatives, in combination with a substrate. The human embryonic germ (EG) cell derivatives are typically human embryoid body-derived cells, for example, LVEC cells or SDEC cells.

The substrate in the aforementioned composition can be collagen I, collagen IV, fibronectin, superfibronectin, laminin, heparan sulfate proteoglycan, entactin, or any combination thereof. Typically, the collagen I is human type I collagen. Other synthetic or biosynthetic adhesion molecules may also be used. Typically a substrate of human origin is used in order to avoid the presence of non-human components in ES cultures, but for purposes other than human therapeutic uses, non-human components may be present. In another embodiment, the substrate is an extracellular matrix, such as that obtained from human embryonic germ (EG) cell derivatives, typically human embryoid body-derived cells. Non-limiting examples include LVEC cells or SDEC cells. In another embodiment, the extracellular matrix is EHS mouse sarcoma basement membrane or human extracellular matrix.

In yet another embodiment, a kit is provided for cultivating human embryonic stem (ES) cells and maintaining the pluripotency thereof, the kit comprising a first container secreted products from human embryonic germ (EG) cell derivatives, a second container of substrate, and instructions for the use thereof. The human embryonic germ (EG) cell derivatives are typically human embryoid body-derived cells, such as but not limited to LVEC cells or SDEC cells. The substrate can be collagen I, collagen IV, fibronectin, superfibronectin, laminin, heparan sulfate proteoglycan, entactin, or any combination thereof. Typically the collagen I is human type I collagen. Other synthetic or biosynthetic adhesion molecules or mixtures may also be used. Typically a substrate of human origin is used in order to avoid the presence of non-human components in ES cultures, but for purposes other than human therapeutic uses, non-human components may be present. In another embodiment the substrate can be an extracellular matrix, such as that obtained from human embryonic germ (EG) cell derivatives, for example, human embryoid body-derived cells (EBD) such as but not limited to cell culture LVEC cells or SDEC cells. The extracellular matrix can be EHS mouse sarcoma basement membrane or human extracellular matrix.

Another embodiment of the invention is a composition comprising pluripotent human embryonic stem (ES) cells and secreted products from human embryonic germ (EG) cell derivatives. The human embryonic germ (EG) cell derivatives are typically human embryoid body-derived cells (EBD) such as but not limited to cell culture LVEC cells or SDEC cells. The composition can further comprise a substrate, such as but not limited to collagen I, collagen IV, fibronectin, superfibronectin, laminin, heparan sulfate proteoglycan, entactin, or any combination thereof. Typically the collagen I is bovine or human type 1 collagen. In another embodiment, the substrate is a synthetic or biosynthetic adhesion molecule or a mixture thereof. Typically a substrate of human origin is used in order to avoid the presence of non-human components in ES cultures, but for purposes other than human therapeutic uses, non-human components may be present. In another embodiment, the composition can include an extracellular matrix. The extracellular matrix can be obtained from human embryonic germ (EG) cell derivatives, typically human embryoid body-derived cells. Non-limiting examples include LVEC cells or SDEC cells. The extracellular matrix can be EHS mouse sarcoma basement membrane or human extracellular matrix.

In another embodiment of the invention, cultured pluripotent human embryonic stem (ES) cells can be obtained by the process of 1) providing a culture medium comprising secreted products from human embryonic germ (EG) cell derivatives, together with a substrate, 2) introducing human embryonic stem (ES) cells thereto; and 3) growing the human embryonic stem (ES) cells therein to produce cultured pluripotent human embryonic stem cells. The human embryonic germ (EG) cell derivatives are typically human embryoid body-derived cells, such as LVEC cells or SDEC cells. The substrate can be collagen I, collagen IV, fibronectin, superfibronectin, laminin, heparan sulfate proteoglycan, entactin, or any combination thereof. Typically, the collagen I is bovine or human type I collagen. In another embodiment, the substrate is a synthetic or biosynthetic adhesion molecule or a mixture thereof. Typically a substrate of human origin is used in order to avoid the presence of non-human components in ES cultures, but for purposes other than human therapeutic uses, non-human components may be present. In another embodiment, the substrate is extracellular matrix, for example, extracellular matrix is obtained from embryonic germ (EG) cell derivatives, typically human embryoid body-derived cells, such as LVEC cells or SDEC cells.

In yet another embodiment of the invention, a method is provided for obtaining a pluripotent human embryonic cell line comprising the steps of 1) isolating human cells from the inner cell mass of a pre-implantation embryo; 2) introducing the cells of (1) into a culture medium comprising a composition of the invention; and 3) growing the human embryonic stem cells derived thereby over several passages in the culture medium, thereby obtaining a human embryonic cell line derived from the pre-implantation embryo. A composition for use in this embodiment can be a composition comprising secreted products from human embryonic germ (EG) cell derivatives, in combination with a substrate. The human embryonic germ (EG) cell derivatives are typically human embryoid body-derived cells, for example, LVEC cells or SDEC cells. The substrate can be collagen I, collagen IV, fibronectin, superfibronectin, laminin, heparan sulfate proteoglycan, entactin, or any combination thereof. Typically, the collagen I is human type I collagen. In another embodiment, the substrate is a synthetic or biosynthetic adhesion molecule or a mixture thereof. Typically a substrate of human origin is used in order to avoid the presence of non-human components in ES cultures, but for purposes other than human therapeutic uses, non-human components may be present. In another embodiment, the substrate is an extracellular matrix, such as that obtained from human embryonic germ (EG) cell derivatives, typically human embryoid body-derived cells. Non-limiting examples include LVEC cells or SDEC cells. In another embodiment, the extracellular matrix is EHS mouse sarcoma basement membrane or human extracellular matrix.

The following sections provide descriptions of each of the components of the present invention. They are intended to be exemplary only and non-limiting, and one of ordinary skill will recognize alternative means for achieving the same result within the spirit of the invention.

Substrates. The aforementioned substrates collagen I (type I collagen), collagen IV (type IV collagen), fibronectin, superfibronectin, laminin, heparan sulfate proteoglycan, entactin, singly or in any combination, are used in an embodiment wherein the substrate is a defined protein or combination of proteins. These proteins are readily available commercially or can be prepared in the laboratory following guidance in the art. Typically human proteins are used in the practice of the invention but this is not so limiting if human therapeutic use is not contemplated.

Extracellular Matrix. Extracellular matrix can be purchased or prepared from cells in accordance with teachings in the art. One example of a mouse extracellular matrix favored in work prior to the invention described herein is EHS mouse sarcoma basement membrane, manufactured by BD Biosciences (San Jose, Calif.) and sold under the name MATRIGEL. A human extracellular matrix is also commercially available from BD Biosciences. Typically, the invention is carried out using type I collagen, which, as has been found by the inventors herein, provides a suitable substrate in combination with the secreted products from embryonic germ (EG) cell derivatives, to permit derivation of embryonic stem (ES) cells as well as propagation while maintaining pluripotency. Typically extracellular matrix of human origin or human derived is used.

In another embodiment, the substrate comprises any synthetic or biosynthetic cell adhesion molecule. Among the substrates described above, fragments and peptides thereof capable of supporting growth of ES cells are further embodiments of the invention. In one embodiment, a peptide comprising the tripeptide RGD is useful as a substrate for the purposes herein described. Typically the substrate is human derived.

Human Embryonic Germ Cell Derivatives. In the practice of the invention, human embryonic germ (EG) cell derivatives may be used as a source of the secreted products that support derivation and growth of ES cells. EG cells can be generated and cultured essentially as described in U.S. Pat. No. 6,090,622. The starting materials for isolating cultured embryonic germ (EG) cells are tissues and organs comprising primordial germ cells (PGCs). For example, PGCs may be isolated over a period of about 3 to 13 weeks post-fertilization (e.g., about 9 weeks to about 11 weeks from the last menstrual period) from embryonic yolk sac, mesenteries, gonadal anlagen, or genital ridges from a human embryo or fetus. Alternatively, gonocytes of later testicular stages can also provide PGCs. In one embodiment, the PGCs are cultured on mitotically inactivated fibroblast cells (e.g., STO cells) under conditions effective to derive EGs. The resulting human EG cells resemble murine ES or EG cells in morphology and in biochemical histotype. The resulting human EG cells can be passaged and maintained for at least several months in culture.

Human Embryoid Bodies and Embryoid Body-Derived Cells. In the practice of the various embodiments of the invention described herein, typically embryoid body-derived cells (EBD) that are derived from embryonic germ cells as mentioned above, are used to provide secreted products. Methods for preparing embryoid body-derived cells are described in U.S. Patent Application Publication No. 2003/0175954, published Sep. 18, 2003, and based on Ser. No. 09/767,421, and incorporated herein by reference in its entirety. Such cells can be derived from human embryoid bodies (EBs), which are in turn produced by culturing EG cells, as described above. Methods for making EBs are described below. Unlike EBs, which are large, multicellular three-dimensional structures, embryoid body-derived cells grow as a monolayer and can be continuously passaged. Although EBD cells are not immortal, they display long-term growth and proliferation in culture. Mixed cell EBD cultures and clonally isolated EBD cell lines simultaneously express a wide array of mRNA and protein markers that are normally associated with cells of multiple distinct developmental lineages, including neural (ectodermal), vascular/hematopoietic (mesodermal), muscle (mesodermal) and endoderm lineages. Mesodermal cells include, for example, connective tissue cells (e.g., fibroblasts) bone, cartilage (e.g., chondrocytes), muscle (e.g., myocytes), blood and blood vessels, lymphatic and lymphoid organs cells, neuronal cells, pleura, pericardium, kidney, gonad and peritoneum. Ectodermal cells include, for example, epidermal cells such as those of the nail, hair, glands of the skin, nervous system, the external organs (e.g., eyes and ears) and the mucosal membranes (e.g., mouth, nose, anus, vaginal). Endodermal cells include, e.g., those of the pharynx, respiratory tract, digestive tract, bladder, liver, pancreas and urethra cells. The growth and expression characteristics of EBD cells reveal an uncommitted precursor or progenitor cells phenotype.

Generating Embryoid Bodies (EBs) and Characterization of EBD Cells. Human embryoid bodies (EBs) form spontaneously in human primordial germ cell-derived stem cell cultures that have been maintained in the presence of leukemia inhibitory factor (LIF) (e.g., human recombinant leukemia inhibitory factor) at about, e.g., 1000 units/ml, basic fibroblast growth factor (bFGF), at about 1 ng/ml, and forskolin at about 10 μM for greater than about one month, and, in some situations, as long as three to six months. EBs are also formed when these factors are withdrawn. Additional factors can be added to enhance or direct this process, including, but not limited to, retinoic acid, dimethylsulfoxide (DMSO), cAMP elevators such as forskolin, isobutylmethylxanthine, and dibutryl cAMP, cytokines such as basic fibroblast growth factor, epidermal growth factor, platelet derived growth factor (PDGF and PDGF-AA) nerve growth factor, T3, sonic hedgehog (Shh or N-Terminal fragment), ciliary neurotrophic factor (CNTF), erythropoeitin (EPO) and bone morphogenic factors. The foregoing list are merely a subset of the known compounds that are useful in this aspect of the invention and are not intended at be limiting.

Moreover, and as will be discussed further below, embryoid body-derived cells used in the practice of the invention include cells as described above as well as those that can be transformed or infected. Guidance for methods of so doing may be found in U.S. Patent Application Publication 2003/0175954. Genetic manipulation, for the purposes of the present invention, include those manipulations that increase the secretion of proteins or other products that support the derivation and proliferation of ES cells or maintain pluripotency thereof.

The following description is an example of the preparation of embryoid body-derived cell lines from embryoid bodies. It is merely exemplary and a skilled artisan will readily find other ways of carrying out this aspect of the invention while not deviating from its spirit. EBs are physically removed from the stem cell culture medium where they are formed (see above), and placed in a calcium and magnesium-free phosphate-buffered saline (PBS). The EBs are then sorted into categories by gross morphology, e.g., cystic or solid. After sorting, the EBs are transferred to a mixture of one mg/ml collagenase and dispase enzyme (Boehringer Mannheim), and incubated for 30 minutes to three hours at 37 C.; during this time they are manually agitated or triturated every about 10 to 30 minutes. Other dissociation treatments can be used, e.g., the individual or combined use of several different types of collagenase, dispase I, dispase II, hyaluronidase, papain, proteinase K, neuraminidase and/or trypsin. Each treatment requires optimization of incubation length and effectiveness; cell viability can be monitored visually or by trypan blue exclusion followed by microscopic examination of a small aliquot of the disaggregation reaction. One collagenase/dispase disaggregation protocol calls for incubation for about 30 minutes at 37 C.; this results in between about 10% and 95% of the EB constituent cells disaggregated into single cells. Large clumps of cell may remain intact.

After disaggregation, one to five mls of growth medium are added to the cells. One exemplary medium comprises EGM2-MV medium (Clonetics/Cambrex) with about 10 to 20% fetal calf serum supplemented with antibiotics, e.g., penicillin and streptomycin. The cell suspension is then centrifuged at about 100 to 500 g for about five minutes. The supernatant is then removed and replaced with fresh growth media. The cells are resuspended and plated into a tissue culture vessel that can be coated with cells or typically a biomatrix. In a typical embodiment, collagen type I is used as the substrate.

EBD cells obtained from 4 to 8 EBs can be resuspended in media, e.g., about three ml media (e.g., RPMI), and plated (e.g., into a 3.5 cm diameter plate) onto a surface that has been coated with a collagen (e.g., human type I collagen). The culture medium is replaced every two to three days. This is a general method that will allow a wide variety of cell types to proliferate.

Cell Culture of EBD Cells to Produce Secreted Products. In one embodiment the invention utilizes EBDs to produce secreted products for deriving, growing and maintaining ES cells in a pluripotent state. As described above, EBD cells can be clonally isolated and are capable of robust and long-term proliferation in culture, where production of secreted products are used for supporting ES cells in accordance with the invention. EBD cells are grown and maintained in culture medium or growth medium. Examples of suitable culture media include EGM2-MV medium as mentioned above, knockout DMEM (from GibcoBRL, Life Technologies), Hepatostim (BD Biosciences) and DMEM medium containing knockout serum (Invitrogen) or plasminate, to name only a few examples.

Secreted products from embryonic germ (EG) cell derivatives is also referred to as “conditioned medium”, a term that refers to a growth medium that is further supplemented by factors derived from media obtained from cultures of cells, in this case, embryonic germ (EG) cell derivatives or embryoid body-derived cells. An effective amount of conditioned medium can be added, e.g., periodically, e.g., daily, to either of these base solutions to prepare human ES derivation or growth media. The term “effective amount” as used herein is the amount of such described factor as to permit a beneficial effect on human ES growth and maintain the pluripotency thereof. Factors or products produced by embryonic germ (EG) cell derivatives that are secreted into the medium can include proteins as well as other cell-derived products. The conditioned medium can be centrifuged to remove cells and other particulates, or filter sterilized, for example, by passage through a 0.22 micrometer filter. Other means of treating or handling the conditioned medium or secreted products described herein to facilely provide growth medium for human ES cells are embraced herein. The secreted products can be maintained in the cold, or frozen, for storage.

Genetic manipulation of the EBD cells useful in the practice of the invention include the production of long-lived cells by telomerase transfection, (see, e.g., U.S. Pat. Nos. 5,863,726; 6,054,575; 6,093,809; WO 98/14592; WO 00/46355). Further, manipulation to increase production of secreted products useful in the practice of the invention is also embraced herein, such as protein that are supportive of ES cell growth.

Pluripotency of the ES cells grown in accordance with the invention may be assessed by any of a number of methods. For example, as shown in an example below, expression of the stem cell marker OCT4 shows the pluripotency of the cultivated cells. Another indicator is the level of alkaline phosphatase, a marker of undifferentiated cells. Other surface markers associated with non-differentiation such as SSEA-4, SSEA-3, TRA-1-60 (ATCC HB-4783) and TRA-1-81 (ATCC HB-4784), and/or the expression of telomerase.

Uses of ES cells derived or propagated as describe herein. The following uses of ES are merely exemplary and non-limiting.

Cell-Based Therapies: Transplantation of ES Cells. The invention also provides methods for growth of unmodified or genetically modified ES cells or their differentiated progeny for use in human transplantations in the fetus, newborns, infants, children, and/or adults. One example of this use is therapeutic supplementation of metabolic enzymes for the treatment of autosomal recessive disorders. For example, production of homogentisic acid oxidase by transplanted ES differentiated cells into the liver could be used in the treatment of alkaptonuria (for review of this disorder, see McKusick, Heritable Disorders of Connective Tissue. 4th ed., St. Louis, C. V. Mosby Co., 1972). Likewise, ornithine transcarbamylase expression could be augmented to treat the disease caused by its deficiency. In another example, glucose-6-phosphate dehydrogenase expression could be augmented in erythrocyte precursors or hematopoietic precursors to allow expression in red blood cells in order to treat G6PD deficiency (favism, acute hemolytic anemnia).

Treatments of some diseases require addition of a composition or the production of a circulating factor. One example is the production of alpha1-antitrypsin in plasma to treat a deficiency that causes lung destruction, especially in tobacco smokers. Other examples of providing circulating factors are the production of hormones, growth factors, blood proteins, and homeostatic regulators.

In another embodiment of the invention, differentiated ES cells obtained or grown as described herein are used to repair or supplement damaged or degenerating tissues or organs. This may require that the cells are first differentiated in vitro into lineage-restricted stem cells or terminally differentiated cells.

Before implantation or transplantation the ES cell obtained or grown as described herein can be genetically manipulated to reduce or remove cell-surface molecules responsible for transplantation rejection in order to generate universal donor cells. For example, the mouse Class I histocompatibility (MHC) genes can be disabled by targeted deletion or disruption of the beta-microglobulin gene (see, e.g., Zijlstra, Nature 342:435-438, 1989). This significantly improves renal function in mouse kidney allografts (see, e.g., Coffinan, J. Immunol. 151:425-435, 1993) and allows indefinite survival of murine pancreatic islet allografts (see, e.g., Markmann, Transplantation 54:1085-1089, 1992). Deletion of the Class II MHC genes (see, e.g., Cosgrove, Cell 66:1051-1066, 1991) further improves the outcome of transplantation. The molecules TAP1 and Ii direct the intercellular trafficking of MHC class I and class II molecules, respectively (see, e.g., Toume, Proc. Natl. Acad. Sci. USA 93:1464-1469, 1996); removal of these two transporter molecules, or other MHC intracellular trafficking systems may also provide a means to reduce or eliminate transplantation rejection. As an alternative to a universal donor approach to histocompatibility, genetic manipulation could be used to generate “custom” MHC profiles to match individual needs.

In addition to manipulating MHC expression, for human transplantation, cells and tissues from ES cells and cell lines grown in accordance with the invention can also be manipulated to eliminate or reduce other cell-surface marker molecules that induce tissue/organ graft rejection. All such modifications that reduce or eliminate allogenic (e.g., organ graft) rejection when employing cells, cell lines (or any parts or derivatives thereof) derived from the cells of the present invention are embodied herein.

Tissue Engineering. The invention provides human cells and methods that can be used to produce or reconstruct a tissue or organ, including in vitro or vivo regeneration, and engineering of artificial organs or organoids. In one aspect, the ES cells grown in accordance with the invention are pre-cultured under conditions that promote generation of a desired differentiated, or restricted, cell lineage. The culture conditions can also be manipulated to generate a specific cell architecture, such as the three-dimensional cellular arrangements and relationships seen in specialized structures, such as neuromuscular junctions and neural synapses, or organs, such as livers, and the like. These conditions can include the use of bioreactor systems to influence the generation of the desired cell type. Bioreactor systems are commonly used in the art of tissue engineering to create artificial tissues and organs. Some bioreactor systems are designed to provide physiological stimuli similar to those found in the natural environments. Others are designed to provide a three-dimensional architecture to develop an organ culture. For example, the compositions (including bioreactors, scaffolds, culture devices, three-dimensional cell culture systems, and the like) and methods described in U.S. Pat. Nos. 6,143,293; 6,121,042; 6,110,487; 6,103,255; 6,080,581; 6,048,721; 6,022,743; 6,022,742; 6,008,049; 6,001,642; 5,989,913; 5,962,325; 5,858,721; 5,843,766; 5,792,603; 5,770,417; 5,763,279; 5,688,687; 5,612,188; 5,571,720; 5,770,417; 5,626,863; 5,523,228; 5,459,069; 5,449,617; 5,424,209; 5,416,022; 5,266,480; 5,223,428; 5,041,138; and 5,032,508; or variations thereof, can be used in conjunction with this invention.

As discussed above, production of cells, tissues and organs for transplantation may require combinations of genetic modifications, in vitro differentiation, and defined substrate utilization of the cells of the invention to generate the desired altered cell phenotype and, if a tissue or organ is to be generated, the necessary three-dimensional architecture required for functionality. For example, a replacement organ may require vasculature to deliver nutrients, remove waste products, and deliver products, as well as specific cell-cell contacts. A diverse cell population will be required to carry out these and other specialized functions, such as the capacity to repopulate by lineage-restricted stem cells.

Further examples of the use of the ES cells obtained or grown in accordance with the invention and their differentiated derivatives include generation of non-cellular structures such as bone or cartilage replacements.

Human ES cells obtained or grown in accordance with the invention can also be implanted into the central nervous system (CNS) for the treatment of disease or physical brain injury, such as ischemia or chemical injury; animal models can also be used to test the efficacy of this treatment, e.g., injection of compounds like 6-hydroxydopamine (6OHAD), or, fluid percussion injury can serve as a model for human brain injury. In these animal models, the efficacy of administration of stem cells of the invention is determined by the recovery of improvement of injury related deficits, e.g., motor or behavioral deficits. Human ES cells obtained in accordance with the invention can also be implanted into the central nervous system (CNS) for the treatment of amyotropic lateral sclerosis (ALS); animal models can also be used to test the efficacy of this treatment, e.g., the SODI mutant mouse model. Human ES cells of the invention can also be implanted into the central nervous system (CNS) for the treatment of Alzheimer's disease; one animal model that can be used to test the efficacy of this treatment is the mutant presenilin I mouse. Human ES cells can also be implanted into the central nervous system (CNS) for the treatment of Parkinson's disease, efficacy of this treatment can be assessed using, e.g., the MPTP mouse model.

Human ES cells grown in accordance with the invention can also be used to treat diseases of cardiac, skeletal or smooth muscles; cells can be directly injected into or near desired sites. The survival and differential of these cells can be determined by monitoring the expression of appropriate markers, e.g, human muscle-specific gene products (see, e.g., Klug, 1996, supra; Soonpaa, Science 264:98-101, 1994; Klug, Am. J. Physiol. 269:H1913-H1921, 1995; implanting fetal cardiomyocytes and mouse ES-derived cells), for exemplary protocols.

Human ES cells grown in accordance with the invention can also be used to treat diseases of the liver or pancreas. Cells can be directly injected into the hepatic duct or the associated vasculature. Similarly, cells could be delivered into the pancreas by direct implantation or by injection into the vasculature. Cells engraft into the liver or pancreatic parenchyma, taking on the functions normally associated with hepatocytes or pancreatic cells, respectively. As with other implantations, cell survival, differentiation and function can be monitored by, e.g., immunohistochemical staining, or PCR, of specific gene products.

Human ES cells of the invention can also be used to treat diseases, injuries or other conditions in or related to the eyes. Cells can be directly injected into the retina, optic nerve or other eye structure. In one aspect, cells differentiate into retinal epithelia, nerve cells or other related cell types. As with other engraftments, cell survival, differentiation and function can be monitored by, e.g., immunohistochemical staining, or PCR, of specific gene products.

Human ES cells of the invention can also be used to treat vascular diseases or other related conditions by repopulation of the vasculature with, e.g., vascular endothelium, vascular smooth muscle and other related cell types. For example, an injured vein or artery is treated by implantation of ES cells of the invention; these cells re-populate the appropriate injured sites in the vasculature. The cells can be implanted/injected into the general circulation, by local (“regional”) injection (e.g., into a specific organ) or by local injection, e.g., into a temporarily isolated region. In an alternative procedure, a reconstructed or a completely new vasculature can be constructed on a biomatrix or in an organotypic culture, as described herein.

Human ES cells of the invention can also be used to repopulate bone marrow, e.g., in situations where bone marrow has been ablated, e.g., by irradiation for the treatment of certain cancers. Protocols for these treatments can be optimized using animal models, e.g., in animals whose endogenous bone marrow has been ablated. EBD cells of the invention can be injected into the circulatory system or directly into the marrow space of such an animal (e.g., a rodent model). Injection of the human cells of the invention would allow for the re-population of bone marrow, as well as engraftment of a wide range of tissues and organs. If the animals are sublethally irradiated, the efficacy of the cells can be monitored by tracking animal survival, as without bone marrow re-population the animal will die. The hematopoietic fate of the injected cells also can be examined by determining the type and amount to human cell colonies in the spleen.

In another aspect, the human ES cells obtained or grown in accordance with the invention can be used in organotypic co-culture. This system offers the benefits of direct cell application and visualization found in in vitro methods with the complex and physiologically relevant milieu of an in vivo application. In one aspect, a section of tissue or an organ specimen is placed into a specialized culture environment that allows sufficient nutrient access and gas exchange to maintain cellular viability.

In using the human ES cells, or differentiated derivatives thereof, of the invention to construct artificial organs or organoids, bioengineered matrices or lattice structures can be populated by single or successive application of these human cells. The matrices can provide structural support and architectural cues for the repopulating cells.

Biosensors and Methods of Screening. ES cells or cell lines obtained or grown in accordance with the invention and cells, tissues, structures and organs derived from them can be used for toxicological, mutagenic, and/or teratogenic in vitro tests and as biosensors. Thus, the invention provides engineered cells, tissues and organs for screening methods to replace animal models and form novel human cell-based tests. These systems are useful as extreme environment biosensors. ES cells or cell lines and cells, tissues, structures and organs derived from them can be used to build physiological biosensors; for example, they can be incorporated in known system, as described, e.g., in U.S. Pat. Nos. 6,130,037; 6,129,896; and 6,127,129. These sensors can be implanted bio-electronic devices that function as in vivo monitors of metabolism and other biological functions, or as an interface between human and computer.

The invention also provides a method for identifying a compound that modulates an ES cell function in some way (e.g., modulates differentiation, cell proliferation, production of factors or other proteins, gene expression). The method includes: (a) incubating components comprising the compound and ES cell(s) grown under conditions described herein, sufficient to allow the components to interact; and (b) determining the effect of the compound on the ES cell(s) before and after incubating in the presence of the compound. Compounds that ES cell function include peptides, peptidomimetics, polypeptides, chemical compounds and biologic agents. Differentiation, gene expression, cell membrane permeability, proliferation and the like can be determined by methods commonly used in the art. The term “modulation” refers to inhibition, augmentation, or stimulation of a particular cell function.

ES Cells as Sources of Macromolecules. The ES cells and cell lines obtained or grown in accordance with the invention can also be used in the biosynthetic production of macromolecules. Non-limiting examples of products that could be produced are blood proteins, hormones, growth factors, cytokines, enzymes, receptors, binding proteins, signal transduction molecules, cell surface antigens, and structural molecules. Factors produced by undifferentiated, differentiating, or differentiated ES cells would closely simulate the subtle folding and secondary processing of native human factors produced in vivo. Biosynthetic production by ES cells and cell lines can also involve genetic manipulation followed by in vitro growth and/or differentiation. Biosynthetic products can be secreted into the growth media or produced intracellularly or contained within the cell membrane, and harvested after cell disruption. Genetic modification of the gene coding for the macromolecule to be biosynthetically produced can be used to alter its characteristics in order to supplement or enhance functionality. In this way, novel enhanced-property macromolecules can be created and pharmaceuticals, diagnostics, or antibodies, used in manufacturing or processing, can be produced. Pharmaceutical, therapeutic, processing, manufacturing or compositional proteins that may be produced in this manner include, e.g., blood proteins (clotting factors VIII and IX, complement factors or components, hemoglobins or other blood proteins and the like); hormones (insulin, growth hormone, thyroid hormone, gonadotrophins, PMSG trophic hormones, prolactin, oxytocin, dopamine, catecholamines and the like); growth factors (EGF, PDGF, NGF, IGF and the like); cytokines (interleukins, CSF, GMCSF, TNF, TGF.alpha., TGF.beta., and the like); enzymes (tissue plasminogen activator, streptokinase, cholesterol biosynthetic or degradative, digestive, steroidogenic, kinases, phosphodiesterases, methylases, de-methylases, dehydrogenases, cellulases, proteases, lipases, phospholipases, aromatase, cytochromes adenylate or guanylate cyclases and the like); hormone or other receptors (LDL, HDL, steroid, protein, peptide, lipid or prostaglandin and the like); binding proteins (steroid binding proteins, growth hormone or growth factor binding proteins and the like); immune system proteins (antibodies, SLA or MHC gene products); antigens (bacterial, parasitic, viral, allergens, and the like); translation or transcription factors, oncoproteins or proto-oncoproteins, milk proteins (caseins, lactalbumins, whey and the like); muscle proteins (myosin, tropomyosin, and the like).

Screens for Culture Media Factors. In another embodiment and use of the invention, ES cells grown in accordance with the teachings herein are used to optimize the in vitro culture conditions for differentiating the cells. High-throughput screens can be established to assess the effects of media components, exogenous growth factors, and attachment substrates. These substrates include viable cell feeder layers, cell extracts, defined extracellular matrix components, substrates which promote three-dimensional growth such as methylcellulose and collagen, novel cell attachment molecules, and/or matrices with growth factors or other signaling molecules embedded within them. This last approach may provide the spatial organization required for replication of complex organ architecture (as reviewed in Saltzman, Nature Medicine 4:272-273, 1998).

### EXAMPLES

The following examples are intended to illustrate but not limit the invention. While they are typical of those that might be used, other procedures known to those skilled in the art may alternatively be used.

### Example 1

### Derivation of Embryoid Germ Cell Derivatives

Human pluripotent germ cell cultures were derived from primordial germ cells, isolated and cultured as described above and in Shamblott et al., Proc. Natl. Acad. Sci. USA 95:13726-13731, 1998. Four genetically distinct human EG cell cultures were selected to represent the range of developmental stages at which human EG cultures can be initiated, with karyotypes as noted LV (46, XX), SL (46, XY), LU2 (46, XY) and SD (46, XX). These cultures were derived and cultured from 5, 6, 7, and 11 week post-fertilization primordial germ cells (PGCs), respectively. Embryoid bodies (EBs) were formed in the presence of leukemia inhibitory factor (LIF, 1000 U/ml), basic fibroblast growth factor (bFGF, 2 ng/ml), forskolin (10 μM) and 15% fetal calf serum (FCS, Hyclone). During routine growth, 1 to 5% of the multicellular EG colonies formed large fluid-filled cystic EBs that were loosely attached to a remaining EG colony or to the fibroblast feeder layer. Approximately 10 cystic EBs from each culture were dissociated by digestion 1 mg/ml in Collagenase/Dispase (Roche Molecular Biochemicals) for 30 min. to 1 hour at 37 C. Cells were then spun at 1000 rpm for 5 min.

EB constituent cells were then resuspended and replated in growth media and human extracellular matrix (Collaborative Biomedical, 5 μg/cm2), and tissue culture plastic. Cells were cultured at 37 C, 5% CO₂, 95% humidity and routinely passaged 1:10 to 1:40 by using 0.025% trypsin, 0.01% EDTA (Clonetics) for 5 min. at 37 C. Low serum cultures were treated with trypsin inhibitor (Clonetics) and then spun down and resuspended in growth media. Cell were cryopreserved in the presence of 50% FCS, 10% dimethylsulfoxide (DMSO) in a controlled rate freezing vessel, and stored in liquid nitrogen. Exemplary cell culture designations LVEC and SDEC are the cells derived as mentioned above (LV, SD) grown on human extracellular matrix (EC).

### Example 2

### Secreted Products Support ES Cell Growth

Many human cell types were screened for their ability to secrete products capable of supporting human ES cell proliferation, as judged by calculating population doubling rate, and percentage of cells expressing OCT4 after 3 passages in a particular environment. All initial studies used the MATRIGEL biomatrix. Almost none of the human cells provided an environment capable of supporting positive population doubling, and if a line was found to support a positive doubling rate, the rate was far below that provided by secreted products from mouse fibroblasts. Surprisingly, it was found that secreted products present in the culture medium of embryoid body-derived cell line LVEC (see Example 1) could support the growth of human ES cells. Culture medium was filter sterilized by passage through a 0.22 micrometer filter before testing, which removed any cells and provided a sterile product. Conditioned media containing secreted protein from LVEC cells allowed for 2.1 doublings, LVEC direct contact allowed for 1.5 doublings, mouse embryo fibroblast conditioned media allowed 2.6 doublings and if no conditioned media or feeder layer support was provided there was 0.3 doublings.

A second embryoid body-derived culture, SDEC, was then tested for the capacity to support human ES cells. SDEC cells have undergone extensive experimental transplantation in mice, rats and a large pre-clinical safety study in African Green monkeys. No animals receiving SDEC cells have ever suffered an adverse effect that could be attributed to the presence or reaction to the human cell transplant. Initially, use of SDEC cells to produce both a suitable extracellular matrix (ECM) for the attachment of hES cells as well as secreting products supporting ES cell growth. ECM preparation was carried out by growing SDEC cells and then lysing them in 20 mM ammonium acetate for 5-10 min. until cells lyse. Not only were the secreted products from SDEC found to support ES growth, but surprisingly it was found that human type I collagen provided a substrate that in combination with the secreted products, supported ES cell growth. This was in distinction to the earlier demonstration that human ES cells cannot grow on type I collagen when provided with mouse embryo fibroblast CM.

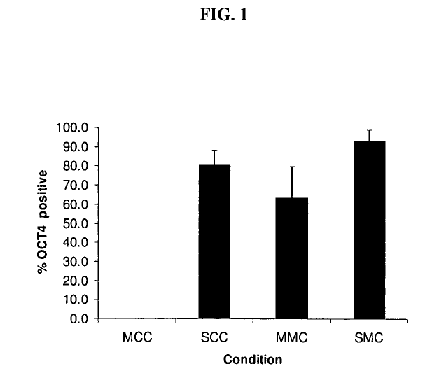

These were continued for 3 passages (disaggregation of cells and replating with ⅓ the number of cells) to evaluate the effects on pluripotency, as determined by % cells that express OCT4. These conditions are; MC (mouse embryo fibroblast CM on type I collagen), SC (SDEC CM on type I collagen), MM (mouse embryo fibroblast CM on Matrigel) and SM (SDEC CM on Matrigel). The enzyme collagenase was used to disaggregate cells between passage and the third letter in the condition name reflects this point. The results are shown in FIG. 1. There is no statistical difference between SCC and SMC, thus showing that products secreted by SDEC permits the use of type I collagen to maintain pluripotency of human ES cells.

In addition to maintenance of pluripotency, a support cell must allow for efficient proliferation in order to expand cell populations. After 3 passages in the four conditions tested as shown in FIG. 1, the population doublings during the third passage were as follows: MCC, 0 (no cells survived); SCC, 2.6; MMC, 2.9; and SMC, 2.4. Thus, secreted products from SDEC are uniquely capable of supporting proliferation on type I collagen. Secreted products from embryonic germ cell derivatives and a type I collagen substrate supported the proliferation of human ES cells.

### Example 3

### Secreted Products Support ES Cell Growth and Maintenance of Pluripotency

Additional studies using several ES cell lines were evaluated for the ability of cells to grow using secreted products described above. High levels of proliferation, and maintenance of pluripotency as determined by OCT4 expression, were demonstrated in WiCell line H1, WiCell line H9 (see http://www.wicell.org/), and HUES 13 cells (see http://http://www.mcb.harvard.edu/melton/hues/). Greater than 95% OCT4 positive cells were shown in 10-20 population doublings. Moreover, high levels of proliferation and maintenance of pluripotency were demonstrated using bovine or human type 1 collagen or superfibronectin as the substrate. Thus, such ES cells can be grown and maintained in an entirely human, cell free medium.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for cultivating human embryonic stem (ES) cells and maintaining the pluripotency thereof comprising growing the human embryonic stem (ES) cells in a culture medium comprising secreted products from human embryonic germ (EG) cell derivatives.

2. The method of claim 2 wherein the human embryonic germ (EG) cell derivatives arc embryoid body-derived cells.

3. The method of claim 2 wherein said human embryoid body-derived cells are LVEC cells or SDEC cells.

4. A method for cultivating human embryonic stem (ES) cells and maintaining the pluripotency thereof comprising growing the human embryonic stem (ES) cells in a culture medium comprising the composition of claim 14.

5. The method of claim 4 wherein the substrate is collagen I, collagen IV, fibronectin, superfibronectin, laminin, heparan sulfate proteoglycan, entactin, or any combination thereof.

6. (canceled)

7. (canceled)

8. (canceled)

9. (canceled)

10. (canceled)

11. (canceled)

12. (canceled)

13. (canceled)

14. A composition for cultivating human stem cells and maintaining the pluripotency thereof comprising secreted products from human embryonic germ (EG) cell derivatives, in combination with a substrate.

15. The composition of claim 14 wherein the human embryonic germ (EG) cell derivatives are human embryoid body-derived cells.

16. The composition of claim 15 wherein said human embryoid body-derived cells arc LVEC cells or SDEC cells.

17. The composition of claim 16 wherein the substrate is collagen I, human type I collagen, collagen IV, fibronectin, superfibronectin, laminin, heparan sulfate proteoglycan, entactin, or any combination thereof.

18. (canceled)

19. The composition of claim 14 wherein the substrate comprises a synthetic or biosynthetic cell adhesion molecule or a mixture thereof.

20. The composition of claim 14 wherein the substrate is extracellular matrix.

21. The composition of claim 20 wherein the extracellular matrix is obtained from human embryonic germ (EG) cell derivatives.

22. The composition of claim 21 wherein the human embryonic germ (EG) cell derivatives are human embryoid body-derived cells.

23. (canceled)

24. (canceled)

25. The composition of claim 19 wherein the human extracellular matrix is obtained from human mesenchymal stem cells, cells derived from human umbilical cord blood or human fibroblasts.

26. The composition of claim 14 wherein the human stem cells are human embryonic stem (ES) cells.

27. A kit for cultivating human embryonic stem (ES) cells and maintaining the pluripotency thereof, the kit comprising a first container secreted products from human embryonic germ (EG) cell derivatives, a second container of substrate, and instructions for the use thereof.

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. A composition comprising pluripotent human embryonic stem (ES) cells and secreted products from human embryonic germ (EG) cell derivatives.

40. The composition of claim 39 wherein said human embryonic germ (EG) cell derivatives are human embryoid body-derived cells.

41. The composition of claim 39 wherein the human embryoid body-derived cells are LVEC cells or SDEC cells.

42. (canceled)

43. (canceled)

44. (canceled)

45. (canceled)

46. (canceled)

47. (canceled)

48. (canceled)

49. (canceled)

50. (canceled)

51. (canceled)

52. Cultured pluripotent human embryonic stem (ES) cells obtained by the process of 1) providing a culture medium comprising the composition of claim 14, 2) introducing human embryonic stem cells thereto; and 3) growing the human embryonic stem cells therein to produce cultured pluripotent human embryonic stem cells.

53. (canceled)

54. (canceled)

55. (canceled)

56. (canceled)

57. (canceled)

58. (canceled)

59. (canceled)

60. (canceled)

61. (canceled)

62. A method for obtaining a pluripotent human embryonic cell line comprising the steps of 1) isolating cells from the inner cell mass of a pre-implantation embryo; 2) introducing the cells of (1) into a culture medium comprising the composition of claim 14, and 3) growing the human embryonic stem cells over several passages in the culture medium, thereby obtaining a human embryonic cell line derived from the pre-implantation embryo.
